(19) United States
(12) Patent Application Publication
(10) Pub. No.: US 2022/0161062 A1
(43) Pub. Date: May 26, 2022

(54) SYSTEMS AND METHODS FOR QUALITY ASSURANCE IN RADIATION THERAPY WITH COLLIMATOR TRAJECTORY DATA

(71) Applicant: DUKE UNIVERSITY, Durham, NC (US)

(72) Inventors: Justus ADAMSON, Durham, NC (US); William GILES, Durham, NC (US); Fang-Fang YIN, Durham, NC (US)

(21) Appl. No.: 17/532,857

(22) Filed: Nov. 22, 2021

Related U.S. Application Data
(60) Provisional application No. 63/116,277, filed on Nov. 20, 2020.

(51) Int. Cl. A61N 5/10; G16H 20/40; G16H 50/20

(52) U.S. Cl. CPC A61N 5/1075; G16H 20/40; A61N 5/103; A61N 5/1045; G16H 50/20

(57) ABSTRACT

Systems and methods are provided for using prior radiotherapy treatment machine parameter trajectory files to determine or predict the machine parameter trajectory at treatment delivery for a new radiotherapy plan, and to quantify the corresponding dosimetric effect of the difference between these machine parameters and the original radiotherapy plan. A pre-treatment quality assurance may thereby be generated that requires no extra beam-on time and provides preemptive insight into the plan quality. The system may include a multi-leaf collimator configured to deliver a treatment plan to a subject and configured to interact with the computer-based algorithm and/or any associated equipment used to perform the quality assurance tasks.

### CROSS-REFERENCE TO RELATED APPLICATIONS

This application claims the benefit of U.S. Provisional Patent Application Ser. No. 63/116,277 filed on Nov. 20, 2020 and entitled “Novel Pre-Treatment IMRT QA Utilizing Trajectory Files with MC Based Dosimetry Analysis,” which is incorporated herein by reference as if set forth in its entirety for all purposes.

### STATEMENT REGARDING FEDERALLY SPONSORED RESEARCH

Not applicable.

### BACKGROUND

Pre-treatment patient specific quality assurance (QA) is an integral part of intensity modulated radiation therapy (IMRT) and volumetric-modulated arc therapy (VMAT) radiotherapy processes, and traditionally includes a plan specific verification measurement and independent dose calculation. Pre-treatment QA measurement traditionally requires that a time intensive measurement be made after completing treatment planning for all IMRT cases. This is a major logistical challenge for implementation of many new technologies in development, such as online adaptive radiotherapy. In addition, traditional pre-treatment IMRT QA requires a high workload for physicists, yet it is poorly correlated with clinical significance and is ineffective at identifying most errors in radiotherapy treatment plans. This has led to a movement towards translating measured dosimetric differences in the IMRT QA phantom geometry to the patient DVH.

The traditional measurement based verification may utilize a detector array either as a field by field or composite measurement, with a comparative analysis using Gamma Index. In a conventional QA approach, a number is assigned and the system either passes or fails by whether the threshold of the assigned number is reached.

Traditional pre-treatment patient specific QA procedure is known to have limitations. Studies have demonstrated that analysis based on Gamma Index for measurement based pre-treatment QA to be a poor predictor of clinically relevant dosimetric errors. This poor correlation persists even for the theoretical case of a fully 3D dose measurement in phantom, indicating the need to translate pre-treatment measurement results back to the patient geometry. This has led to development of methods to translate pre-treatment verification measurements back to the patient dose volume histogram (DVH).

Another limitation of traditional measurement based pre-treatment patient specific QA is incompatibility with emerging online adaptive radiotherapy (OART) techniques. The ability to adapt the treatment according to patient's current anatomy in OART offers unique opportunities to improve therapeutic ratio at various sites and target coverage and reduce dose to organs-at-risk (OARs). Clinical implementation of OART is growing but still limited in part due to the difficulty in achieving real-time efficiency of the treatment planning and delivery process, including the patient-specific QA (PSQA).

Artificial intelligence (AI) has been explored to enhance the pre-treatment patient specific QA process. Algorithms to predict Gamma Index passing rates from IMRT QA such as a weighted Poisson regression with Lasso regularization have been proposed along with those that have incorporated treatment plan characteristics and routine linear accelerator quality control test results into the prediction algorithm. Various machine learning algorithms have been proposed to predict the Gamma Index passing rates for IMRT and VMAT QA with various detector technologies.

There are, however, limitations to this type of AI prediction of outcomes from IMRT and VMAT QA. For instance, the AI model is used to predict the passing rate (typically Gamma Index) for a pre-treatment measurement; hence the AI based prediction will still be subject to the same limitations of the original measurement, namely, being a poor predictor of clinically relevant dosimetric errors. Two factors that contribute to this poor correlation include (1) limitations in the measurement being propagated into the comparative measure, and more importantly, (2) an inherent difference between dose agreement in phantom and dosimetric effect in the clinical plan. An example of the first of these is poor scatter modeling at the detector edge affecting the passing rate, which is then also reflected in the AI model. Regarding the second, while this limitation can be mitigated for a full pre-treatment measurement by translating the measurement results back to the patient geometry, this is not possible with the AI prediction since the final comparative measure is only predicted per field (as opposed to per detector), thus it does not provide sufficient information to reconstruct the clinical dose effect.

Some commercial products have also recently been developed for patient specific trajectory file analysis. The main features of these software packages are recalculating the dose delivered to the patient using the recorded trajectory file for plan specific pre-treatment QA and in-vivo monitoring. However, for many clinical users it remains largely unclear how and if this software fits into the clinical workflow. Many clinics are hesitant to replace their pre-treatment QA procedures with trajectory-file analysis, because conventional trajectory file based pre-treatment IMRT QA requires equal or nearly equal workload as measurement based pre-treatment QA, but without providing a physical and independent measurement. Furthermore, studies have shown that the actual MLC position can vary from the MLC position recorded in the trajectory file, and it remains to be shown that the MLC trajectories in the first fraction will be representative of subsequent fractions. In-vivo monitoring, in which delivered dose to the patient is recalculated using trajectory files from each fraction, adds to the workload with nebulous benefit, since this has not been monitored historically. It is still unclear for which types of cases (if any) the dosimetric effect of positional discrepancies recorded by the trajectory files are significant enough to warrant this additional analysis.

Thus there remains a need for a trajectory file analysis that does not rely solely on the trajectory file in order to capture the total delivery error and for a prediction model for machine parameters that is capable of using DVH based metrics. There is also an ongoing need for an efficient, pre-treatment QA technique that can be carried out offline for rapid turnaround, while still incorporating an independent measurement of MLC position accuracy.

### SUMMARY OF THE DISCLOSURE

The present disclosure addresses the aforementioned drawbacks by providing systems and methods for using prior machine parameter trajectory files to determine or predict the machine parameter trajectory at treatment delivery for a new radiotherapy plan. This provides for a pre-treatment QA that requires no extra beam-on time and provides preemptive insight into the plan quality. In some configurations, a system for IMRT pre-treatment QA is provided. The system may include a computer-based algorithm capable of performing the methods described in the present disclosure. The system may include a multi-leaf collimator configured to deliver a treatment plan to a subject and configured to interact with a computer-based algorithm and/or any associated equipment used to perform IMRT QA tasks. In some configurations, differences between the actual machine parameters and values recorded in the trajectory file are compensated via analysis of routine machine QA tests. In some configurations, differences between calculated and actual dose due to limitations in the beam model, as well as potential variations in machine parameters at treatment delivery are compensated via a plan robustness analysis.

In one configuration, a method is provided for performing a quality assurance (QA) test of a radiation therapy system. The method includes generating a first radiotherapy treatment plan for irradiating a portion of a subject using the radiation therapy system. The method also includes subjecting the first radiotherapy treatment plan to a trained machine parameter determination model to generate predicted machine parameters for the radiation therapy system for delivery of a treatment plan. The method also includes generating a second radiotherapy treatment plan based on the predicted machine parameters. The method also includes determining a dose effect to the subject based on the second radiotherapy treatment plan.

In one configuration, a system is provided for performing a quality assurance (QA) test of a radiation therapy system. The system includes a radiotherapy system configured to provide radiation to a portion of a subject, and a computer system. The computer system is configured to generate a first radiotherapy treatment plan for irradiating the portion of the subject using the radiation therapy system. The computer system is also configured to subject the first radiotherapy treatment plan to a trained machine parameter determination model to generate predicted machine parameters for the radiation therapy system for delivery of a treatment plan. The computer system is also configured to generate a second radiotherapy treatment plan based on the predicted machine parameters. The computer system is also configured to calculate a dose effect to the subject based on the second radiotherapy treatment plan.

In one configuration, a method, a system, or a computer-readable medium is provided as described herein, with the addition that differences between the actual machine parameters and values recorded in the trajectory file are compensated in the second radiotherapy plan by comparing machine parameters measured in routine machine QA tests and those recorded in trajectory files.

In one configuration, a method, a system, or a computer-readable medium is provided as described herein, with the addition that differences between calculated and actual dose due to limitations in the beam model are quantified using a plan robustness analysis. As an example, this plan robustness analysis may consist of a dosimetric analysis after re-calculating the dose using various beam models in which random perturbations have been introduced in the beam model parameters to account for the expected uncertainties in these values.

In one configuration, a method, a system, or a computer-readable medium is provided as described herein, with the addition that differences in actual and calculated dose due to potential variations in machine parameters at treatment delivery are quantified using a plan robustness analysis. In this case the plan robustness analysis may consist of a dosimetric analysis after introducing random perturbations to the treatment plan based on the expected uncertainty and reproducibility of these values.

A non-transitory computer-readable medium is also disclosed and includes instructions that, when executed by a processor, cause the processor to execute the methods disclosed herein.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a flowchart for a non-limiting example workflow for a patient-specific quality assurance method.

FIG. 2 is a flowchart of non-limiting example steps for training a machine learning model for determining machine parameters for radiotherapy delivery to a subject.

FIG. 3 depicts graphs of the performance for the models for the validation dataset in a non-limiting example.

FIG. 4 depicts graphs of the correlation of predicted and actual change in dosimetric indices due to discrepancies in machine parameters at delivery in a non-limiting example.

FIG. 5 is a block diagram of a radiation therapy system that may be used in accordance with the present disclosure.

FIG. 6 is a block diagram of a non-limiting example system for determining or predicting the machine parameter trajectory for a radiotherapy treatment plan and generating a machine parameter model.

FIG. 7 is a block diagram of non-limiting example hardware that can be used to implement the systems and methods in accordance with some embodiments of the disclosed subject matter.

### DETAILED DESCRIPTION

Systems and methods are provided for using prior radiotherapy treatment machine parameter trajectory files to determine or predict the machine parameter trajectory at treatment delivery for a radiotherapy plan. This provides for a “virtual” pre-treatment QA that requires no extra beam-on time and provides preemptive insight into the plan quality. In some configurations, a system for IMRT pre-treatment QA is provided. The system may include a computer-based algorithm capable of performing the methods described in the present disclosure. The system may include a multi-leaf collimator configured to deliver a treatment plan to a subject and configured to interact with the computer-based algorithm and/or any associated equipment used to perform IMRT QA tasks.

Pre-treatment QA may result in providing a user with information needed to modify a treatment plan for a subject, such as when the QA analysis indicates that the previous plan would exceed the capabilities of the radiotherapy system, or not deliver the radiation dose as intended. The QA analysis may also result in identifying a mistake in a plan, which may be correct for future treatment fractions. The QA analysis may also identify needed machine maintenance, such as a need to re-calibrate the system to correct for MLC position inaccuracies.

A trajectory file may be compared directly to an DICOM-RT file rather than relying solely on the trajectory file itself in order to capture the total delivery error. In some configurations, a prediction model may be used to account for total delivery error. The prediction model may be a machine learning or artificial intelligence (AI) powered model for prediction of radiotherapy treatment machine parameters that provide a framework for a virtual patient-specific pre-treatment QA that is capable of calculating DVH based metrics.

A virtual pre-treatment patient-specific QA strategy may be used to determine parameters, such as radiotherapy treatment machine parameters at delivery of radiotherapy to a subject. Discrepancies in machine parameters that occur at treatment delivery for a new treatment plan may be determined or predicted using the trajectory files acquired from prior treatments or from previous subjects. The determined or predicted machine parameters may be incorporated into a determined DICOM radiotherapy (RT) plan and dose may be determined based on patient geometry in conjunction with an independent Monte Carlo dose calculation to directly determine the clinical dosimetric effect.

The systems and methods of the present disclosure may be compatible with OART and may provide for direct feedback regarding clinical dosimetric effects. Determinations or predictions may be validated after the first (and/or each subsequent) treatment fraction by comparison with the trajectory file. The systems and methods may be combined with existing log file analysis and may include independent dose calculation QA strategies. For the independent dose calculation, an independent check of MU calculation may be performed within 48 hours of the start of treatment and a second check dose calculation may be used to verify IMRT treatment deliveries.

One aspect of the present disclosure provides a machine learning prediction model that includes a first model for uncertainty introduced from converting an ideal radiotherapy plan into a deliverable trajectory of machine parameters, a second model for uncertainty in delivering a trajectory of machine parameters, and a third model comprising the first model and the second model. Another aspect of the present disclosure provides a method of patient specific pre-treatment quality assurance (QA) for radiation therapy, using the machine learning prediction model disclosed herein. Another aspect of the present disclosure provides a system, comprising a computing platform and the machine learning prediction model disclosed herein. Another aspect of the present disclosure provides a non-transitory computer-readable medium having stored thereon instructions that, when executed by a processor, cause the processor to execute the methods disclosed herein.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

The term “effective amount” or “therapeutically effective amount” refers to an amount sufficient to effect beneficial or desirable biological and/or clinical results.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. In some embodiments, the subject comprises a human who is undergoing a procedure using a system and/or method as prescribed herein.

Unless otherwise defined, all technical terms used herein have the same meaning as commonly understood by one of ordinary skill in the art to which this disclosure belongs.

In one aspect, the present disclosure provides a radiotherapy patient specific pre-treatment quality assurance (QA) technique. The disclosed systems and methods incorporate physical measurements of multileaf collimator (MLC) position accuracy and translates delivery discrepancies to the dose-volume histogram (DVH) in real time. The technique can advantageously be carried out immediately (e.g., during a radiation procedure while the subject is on the table) in order to support online adaptive radiotherapy technologies.

In one aspect of the present disclosure, the proposed technique comprises a machine learning prediction model that is trained using previous trajectory files for a specific radiotherapy treatment machine. The model predicts the discrepancy in radiotherapy treatment machine moving components (e.g., MLCs, gantry angle), which can be recorded in the trajectory file for a new treatment plan. The model can be updated on an ongoing basis to keep an up-to-date model for each radiotherapy treatment machine.

In one aspect, the prediction model can be created for separate components: the uncertainty introduced from converting an ideal radiotherapy plan from the treatment planning system into a deliverable trajectory of machine parameters, the uncertainty in delivering the trajectory of machine parameters, and the like. These components can be predicted independently of each other and the uncertainties can then be combined into a final model. It is noted that, while there are some existing prediction models, conventional models do not propose predicting these components independently.

In another aspect, the method includes a single field QA plan that is designed to be a rigorous test of radiotherapy treatment machine MLC speed and positioning capability. This QA plan can be designed so that the accuracy of MLC positioning can be quantified using an integrated EPID image, similar to conventional QA tests but more comprehensive in nature. Automated code can be used to analyze the results of the QA plan and detect MLC discrepancies that are not identified by the trajectory file, but which are known to be systematic and usually indicative of a failing leaf component. These measured uncertainties are recorded in a database that tracks the leaf specific uncertainty. This database can be updated regularly, such as by daily, weekly, or monthly delivery of the QA plan. Because this test is carried out using a single field for the machine, it advantageously does not have to be carried out on a patient-by-patient basis. Thus, measurement based component can be carried out a-priori, enabling a measurement based QA that is applicable to online adaptive radiotherapy.

In another aspect, the machine learning prediction model may be combined with the measured MLC uncertainty to construct a plan specific pre-treatment intensity-modulated radiation therapy (IMRT) QA algorithm. For a new IMRT plan, the algorithm predicts delivery discrepancies, which incorporate predicted discrepancies in the trajectory file and measured uncertainties in MLC positioning accuracy. The dosimetric effect on the patient treatment plan is then calculated and displayed in a treatment planning system or independent dose calculation software.

Unlike conventional solutions, the present disclosure may utilize prior trajectory files to predict the trajectory for a new treatment plan. This allows for a “virtual” pre-treatment QA which requires no extra beam on time and still provides preemptive insight into the plan quality. The results of this pre-treatment QA can then be verified by comparing to the trajectory file acquired during the first treatment fraction. Furthermore, concerns by physicists regarding the potential for differences between the actual and recorded MLC positions in the trajectory file are allayed by incorporating into the pre-treatment QA algorithm information regarding MLC accuracy from an independent EPID based measurement. This measurement can be incorporated into routine QA tests which are already being delivered, thus providing a measurement based pre-treatment QA with vastly improved efficiency over other pre-treatment QA methods.

Another embodiment of the present disclosure provides a system for IMRT pre-treatment QA. The system comprises a computer-based algorithm capable of performing the method described hereinabove. The system optionally comprises a multi-leaf collimator configured to interact with the algorithm and/or any other associated equipment necessary to perform IMRT QA tasks, which will be evident to those of skill in the art and which are not described in further detail herein.

The systems and methods in accordance with the present disclosure can be implemented in hardware, software, firmware, or combinations of hardware, software and/or firmware. In some examples, the systems described in this specification may be implemented using a non-transitory computer readable medium storing computer executable instructions that when executed by one or more processors of a computer cause the computer to perform operations. Computer readable media suitable for implementing the systems described in this specification include non-transitory computer-readable media, such as disk memory devices, chip memory devices, programmable logic devices, random access memory (RAM), read only memory (ROM), optical read/write memory, cache memory, magnetic read/write memory, flash memory, and application-specific integrated circuits. In addition, a computer readable medium that implements a system described in this specification may be located on a single device or computing platform or may be distributed across multiple devices or computing platforms.

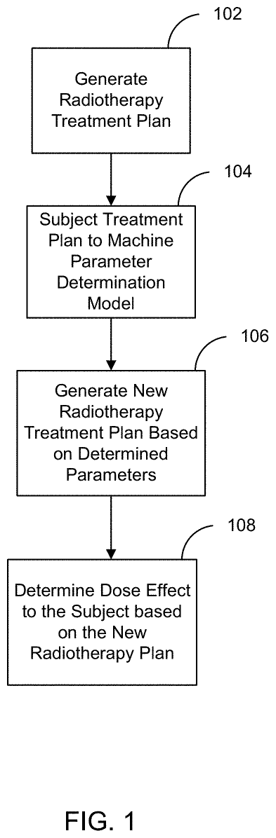

Referring to FIG. 1, a flowchart for a non-limiting example workflow for a patient-specific QA method is shown. A radiotherapy treatment plan may be generated and approved for clinical delivery at step 102. The radiotherapy treatment plan may be a DICOM-RT plan that may be exported from a treatment planning system and imported into a machine learning or AI prediction system. The treatment plan may be subjected to a machine parameter determination model at step 104. Non-limiting example machine parameters include MLC velocity, MLC acceleration, MLC bank, control point number, monitor unit fraction, dose rate, gravity vector, gantry velocity, gantry acceleration (such as for VMAT), and the like.

In some configurations, the machine parameter determination may include using a machine learning or AI model that was previously trained using trajectory files from prior patients to determine or predict machine parameters at delivery for the treatment plan. A new radiotherapy treatment plan may be generated based on the determined parameters at step 106. The new treatment plan may be used to determine the dose effect to the subject at step 108. In some configurations, determining the dose delivery to the subject may be based on a subject's geometry and/or a DVH-based analysis in conjunction with an independent dose calculation, such as a Monte Carlo independent dose calculation. The robustness of the machine parameter prediction model for the treatment plan may be directly validated using the trajectory files that are passively acquired at the first and subsequent treatment fractions for the treatment plan of the subject.

A dataset consisting of radiotherapy treatment plans, such as DICOM-RT plans, and trajectory or log files may be generated from previously treated subjects as a training set for the machine parameter determination model. A portion of the dataset may also be used as a testing set to test the machine parameter determination model. Independent prediction models may be generated for IMRT and VMAT applications. After training and validation of the model, the model may be applied clinically to predict the dosimetry at treatment delivery for new subjects, and may be validated by comparison to results from both the conventional measurement based pre-treatment QA and the trajectory files at treatment delivery.

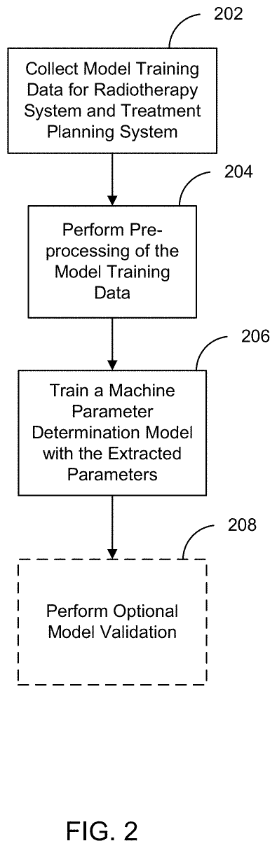

Referring to FIG. 2, a flowchart of non-limiting example steps for training a machine learning or AI model for determining machine parameters for radiotherapy delivery to a subject is shown. Model training data may be generated at step 202. The model training data may include trajectory files and DICOM-RT plans from prior treatments of the subject, or from prior subjects. In a non-limiting example, the model training data may be randomized into one set for training/testing (80% of fields) and one set for post-training validation (20% of fields). The model training data may be pre-processed at step 204 and machine parameters from the radiotherapy system may be extracted. Non-limiting example machine parameters include MLC velocity, MLC acceleration, MLC bank, control point number, monitor unit fraction, dose rate, gravity vector, gantry velocity, gantry acceleration (such as for VMAT), and the like.

Pre-processing and extraction may include extracting radiotherapy treatment machine parameters for each control point to serve as the input to the machine parameter determination model. Pre-processing may also include where the difference between the actual and planned positions per MLC leaf are determined, which may serve as the output of the machine parameter determination model. The machine parameter determination model may be trained with the extracted machine parameters at step 206. The machine parameter determination model may be trained iteratively using a training and testing dataset. In a non-limiting example, a dataset may be split into an model training and testing datasets. Separate models may be generated for IMRT and VMAT. Optionally after the model has been trained or finalized, the model may be evaluated or validated using a testing dataset at step 208. The resulting machine learning or AI model may be used in step 104 of FIG. 1.

Data for training the model may include trajectory files and DICOM-RT plans, such as DICOM-RT plans for IMRT fields and/or VMAT fields acquired from radiotherapy treatment machines. In some configurations, different models of radiotherapy treatment machines may be used to provide the plans used for training the model. A single combined machine parameters model may then be created for the different models of radiotherapy treatment machines.

Examples of suitable radiotherapy treatment machines for use in the present disclosure include, but are not limited to, a linear accelerator, a Cobalt-60 machine, or a combination thereof.

Data pre-processing and machine parameters model construction may be carried out using an appropriate computer system, such as a workstation coupled to the radiotherapy system. The computer system may also be used for data validation and analysis. Radiotherapy treatment machine parameters may be extracted at each control point that serve as input to the machine parameters model including: MLC position, MLC velocity, MLC acceleration, MLC bank, control point number, monitor unit fraction, dose rate, gantry angle, gravity vector (defined as the gravitational pull on each MLC, and which is dependent on gantry angle), and in the case of VMAT, gantry velocity and gantry acceleration, and the like. The computer system may record the machine parameters, such as radiotherapy treatment machine system parameters, at defined time intervals. In a non-limiting example, the time interval may be every 20 ms. The machine parameters may be stored in a trajectory file in binary format, and may be extracted using the computer system, which may include a log analyzer. The machine parameters may be interpolated to the integer control point values found in the DICOM-RT plan.

The machine parameters may be determined from the DICOM-RT file and/or from the trajectory file as some parameters used for the machine parameters model may not be defined within the DICOM-RT file (such as dose rate, MLC velocity, MLC acceleration, gantry velocity, & gantry acceleration). Parameters not defined within the DICOM-RT file may instead be determined by accounting for machine limitations of the radiotherapy treatment machine, such as by measuring the MLC or gantry response time to create a new MLC/gantry position respectively. The output from the machine parameters model may be the discrepancy per MLC leaf between the recorded position at treatment delivery (e.g., actual position) recorded by the trajectory file and the original position recorded in the DICOM-RT plan (e.g., planned position). When the model is used in a QA analysis, as depicted in FIG. 1, the model output may provide the value to be added to the original DICOM-RT planned position to arrive at the predicted “actual” values at treatment delivery.

The machine parameter models may be generated using machine learning, AI, and the like. The target response of the models may be set to the MLC delivery error and the feature variables of the models may be set to the machine parameters described above. During each iteration of the model training process, the training data may be split into training and testing sub-groups. Any appropriate model may be used, including linear regression, decision trees, advanced decision-tree, ensemble algorithms (e.g., boosting, bagging), and neural networks, such as a convolutional neural network, and the like. In some configurations, the machine parameter model may be formed from a combination of models. In a non-limiting example, the boosted-tree model randomly selects and trains a data subset to create a decision tree, and the other subsets are trained sequentially using a previously trained decision tree. In a non-limiting example, the bagged tree model may independently train each subset into a decision tree, with the result being the average of all predictions from different trees.

After the final machine parameter models have been selected and trained, the accuracy of the model at predicting machine parameters at treatment delivery may be quantified, such as by using the subset of the data that was allocated for validating the model and which had not been utilized in training the model. Model accuracy may be assessed by comparing predicted MLC positions from the machine parameter model to the actual MLC positions determined from the trajectory file relative to the DICOM-RT file.

The machine parameter model may be implemented clinically following a workflow, such as that depicted in FIG. 1. The independent dose calculation may include a Monte Carlo based dose calculation. The dose calculation may be performed on the same computer system as that used clinically for a pre-treatment independent volumetric dose calculation. The Monte Carlo dose determination may include using the voxel Monte Carlo (VMC) family of codes (such as VMC++ and XVMC), and may include a virtual source model.

The Monte Carlo independent dose calculation may be carried out on the original DICOM-RT plan, using the machine or machine parameters derived from the machine parameter prediction model, as well as the machine parameters recorded from the trajectory file at treatment delivery. Dosimetric statics may be tabulated for all gross tumor volumes (GTVs), clinical tumor volumes (CTVs), and planning target volumes (PTVs) and for select organs at risk (OARs). In a non-limiting example, dose statistics may include the dose received by 99%, 95%, and 1% of the volume (D99%, D95%, D1%), as well as the mean dose (Dmean) and the percent of volume receiving the prescription dose (V100%).

Conventional pre-treatment QA techniques rely upon QA results such as Gamma Index or dose difference. In accordance with the present disclosure, a virtual pre-treatment QA technique that utilizes a predictive machine parameter model provides for the dosimetric effect on the patient anatomy to be determined directly using a prediction model based on radiotherapy treatment machine trajectory files. An advantage of using a predictive machine parameter model may be in overcoming the poor correlation of Gamma Index with clinically relevant dosimetric errors, as would be found in conventional approaches. Another advantage of using a predictive machine parameter model includes the preponderance of data for training the model. When predicting Gamma Index, each conventional, manual QA delivery and analysis provides a single data point for training, whereas radiotherapy treatment machine trajectory files are recorded passively, and each delivery includes hundreds to thousands of data points that may be used for training in accordance with the present disclosure. With trajectory files it is possible to continuously retrain the model on a regular, even daily, basis as new trajectory files are recorded, which is not possible with conventional approaches.

Trajectory files may include the machine parameters recorded by the radiotherapy system or radiotherapy treatment machine itself. Increased frequency of a QA procedure in accordance with the present disclosure may mitigate errors associated with determining the true position of the MLC, and beam characteristics such as beam symmetry and variations in output per monitor unit.

### Non-Limiting Example

In a non-limiting example, 120 unique IMRT fields (877,098 control points) and 206 unique VMAT fields (1,208,442 control points) were acquired from four separate linear accelerators. Plans were acquired and prepared in the Eclipse treatment planning system v15.1 (Varian Medical Systems, Palo Alto, Calif.). Two models of linear accelerators were used to deliver the plans: TrueBeam STx (Varian Medical Systems, Palo Alto, Calif.) equipped with HD120 MLC™ High-Definition Multileaf Collimator (HDMLC) (Varian Medical Systems, Palo Alto, Calif.) and TrueBeam (Varian Medical Systems, Palo Alto, Calif.) equipped with Millennium™ 120 Leaf MLC (Varian Medical Systems, Palo Alto, Calif.).

Data pre-processing and AI model construction were primarily carried out using Python 3.7 (Python Software Foundation, Wilmington, Del.). MATLAB R2019a (The MathWorks, Inc., Natick, Mass.) was also used for data validation and analysis. Linear accelerator machine parameters extracted at each control point that serve as input to the prediction model included: MLC position, MLC velocity, MLC acceleration, MLC bank, control point number, monitor unit fraction, dose rate, gantry angle, gravity vector (defined as the gravitational pull on each MLC, and which is dependent on gantry angle), and in the case of VMAT, gantry velocity and gantry acceleration. The TrueBeam™ system recorded the linear accelerator machine parameters every 20 ms which are stored in a trajectory file in binary format, and which were extracted using a Python 3.7 (Python Software Foundation, Wilmington, Del.) script in conjunction with the log analyzer module in Pylinac. These values were then interpolated to the integer control point values found in the DICOM-RT plan. When the machine parameters were calculated from the DICOM-RT file rather than from the trajectory file, some parameters used for the prediction model were not explicitly defined within the DICOM-RT file (such as dose rate, MLC velocity, MLC acceleration, gantry velocity, & gantry acceleration), but were instead calculated accounting for machine limitations of the linear accelerator. The output from the prediction model was the discrepancy per MLC leaf between the recorded position at treatment delivery (actual) recorded by the trajectory file and the original position recorded in the DICOM-RT plan (planned). When training the model this value was calculated and extracted from the DICOM-RT and trajectory files. When the prediction model was used in the QA workflow, its output provided the value to be added to the original DICOM-RT planned position to arrive at the predicted “actual” values at treatment delivery.

The AI models were built using the Scikit-learn toolkit. The target response of these models was set to the MLC delivery error and the feature variables of the models were set to the machine parameters described above. During each iteration of the model training process the training data was randomly split into training (80%) and testing (20%) sub-groups. A number of prediction models were evaluated, including linear regression, decision trees, ensemble algorithms (e.g., boosting, bagging), and neural networks. The final models utilized one of two advanced decision-tree models, a boosted tree and a bagged tree. Both models began with separating the input data into multiple subsets. The boosted-tree model randomly selected and trained a subset to create a decision tree, and the other subsets were trained sequentially using the previously trained decision tree. The bagged tree independently trained each subset into a decision tree, with the result being the average of all predictions from different trees.

After the final AI models were selected and trained, their accuracy at predicting machine parameters at treatment delivery was quantified for the 20% of plans that were allocated for validating the model and which had not been utilized in training the model. Model accuracy was assessed by comparing predicted MLC positions from the AI model to the actual MLC positions determined from the trajectory file relative to the DICOM-RT file.

After the AI model was selected, trained, and validated, it was implemented clinically. For the independent dose calculation, a Monte Carlo based dose calculation was employed, which is the same software and dose calculation used clinically for a pre-treatment independent volumetric dose calculation (SciMoCa Version 1.5.1.2890, Scientific-RT, Munich Germany). The Monte Carlo dose engine shared fundamental concepts with the voxel Monte Carlo (VMC) family of codes (such as VMC++ and XVMC) along with a virtual source model. Commissioning of the dose engine for clinical use was carried out previously, which included use of independent beam data.

The clinical workflow was carried out for 7 IMRT treatment plans (breast x1, lung SBRT x1, head and neck x3, prostate & lymph nodes x1, gynecological pelvis & lymph nodes x1) with a total of 61 IMRT fields, as well as 10 VMAT plans (single isocenter multi-target radiosurgery x10) with a total of 35 VMAT arcs. The Monte Carlo independent dose calculation was carried out on the original DICOM-RT plan using the machine parameters derived from the AI prediction model, as well as the machine parameters recorded from the trajectory file at treatment delivery. Dosimetric statics were tabulated for all gross tumor volumes (GTVs), clinical tumor volumes (CTVs), and planning target volumes (PTVs) and for select organs at risk (OARs). Dose statistics included the dose received by 99%, 95%, and 1% of the volume (D99%, D95%, D1%), as well as the mean dose (D mean) and the percent of volume receiving the prescription dose (V100%). In order to facilitate combined analysis of the IMRT plans from a variety of treatment sites, a single set of ring structures were used for the OARs. These included ring structures located 0-3 mm from the PTV edge (Ring0-3 mm), 3-6 mm from the PTV edge (Ring3-6 mm), and 6-9 mm from the PTV edge (Ring6-9 mm). Dose statics recorded for the ring structures were the same as those for the PTVs. The OAR used for the radiosurgery VMAT cases was the brain, with tabulated dose statistics for the percent of the brain receiving 12Gy (V12Gy), and percentage of the brain receiving 20%, 50%, 75%, and 100% of the prescription dose (V20%, V50%, V75%, V100%).

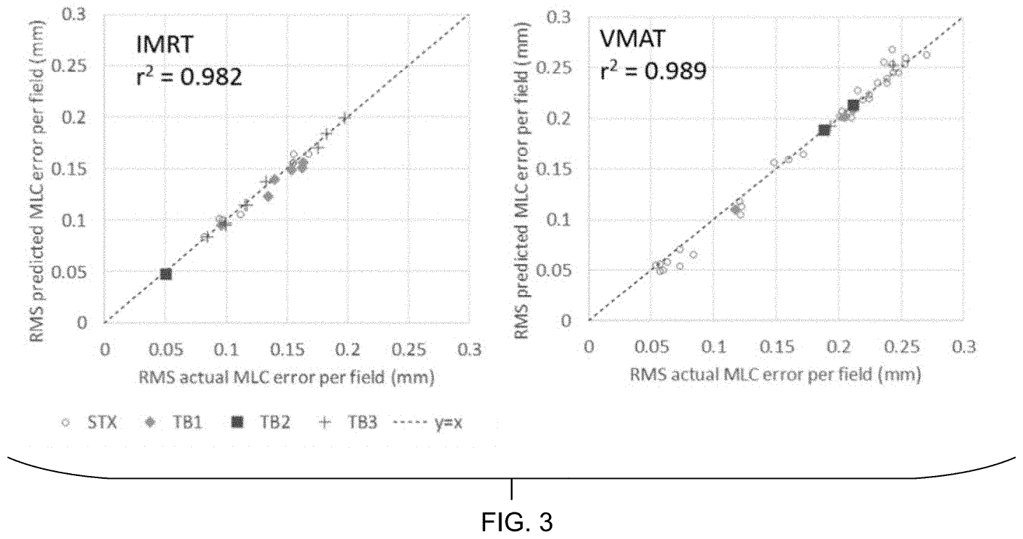

Referring to FIG. 3, graphs of the performance for the models for the validation dataset in the non-limiting example are shown. The final selected prediction model was a boosted tree for IMRT and a bagged tree for VMAT. For IMRT, the average coefficient of determination (r2) for each individual field when comparing the predicted and recorded delivery error for each control point was 0.987±0.012 [0.953, 0.997]. For the VMAT model, this value was 0.895±0.095 [0.453, 0.964]. When comparing the root mean square (RMS) of the MLC delivery error per field, r2 of the predicted vs. actual RMS values was 0.982 for IMRT and 0.989 for VMAT.

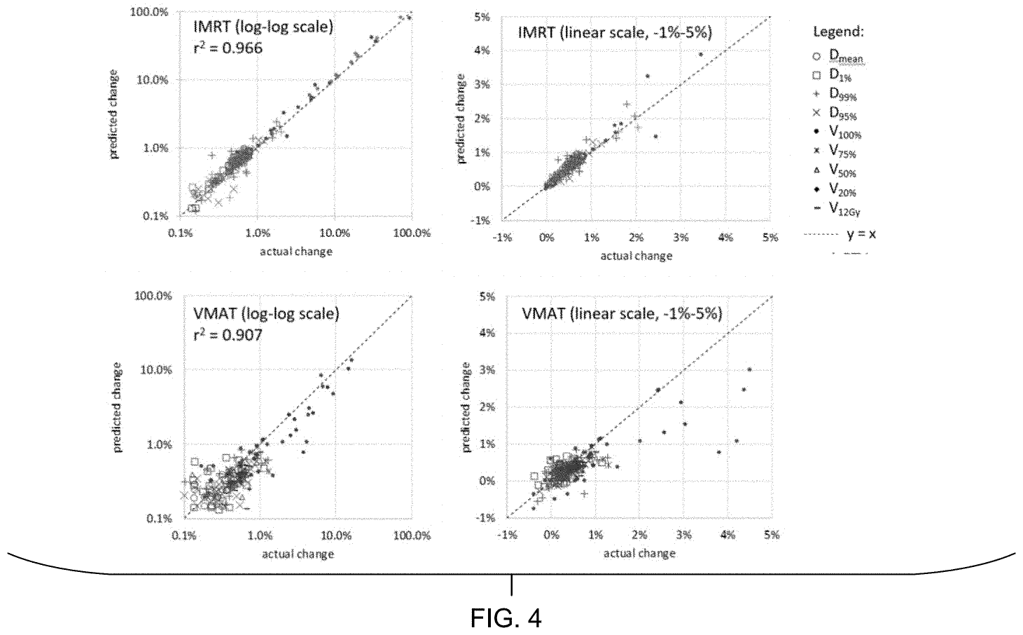

Referring to FIG. 4, graphs of the correlation of predicted and actual change in dosimetric indices due to discrepancies in machine parameters at delivery (r2=0.966 for IMRT, r2=0.907 for VMAT) are shown. The pre-treatment simulated QA procedure was carried out for the 7 IMRT and 10 VMAT plans. Dose for all plans (original, trajectory file, AI prediction) was re-calculated with the Monte-Carlo independent dose calculation software using the patient's original CT and structure set. For this set of patients and set of dosimetric indices, the sensitivity (true positive rate) and specificity (true negative rate) of detecting a 5% change in any of the dosimetric indices on the patient anatomy was 100% and 99.4% respectfully, for IMRT, and 71.4% and 100% respectfully, for VMAT.

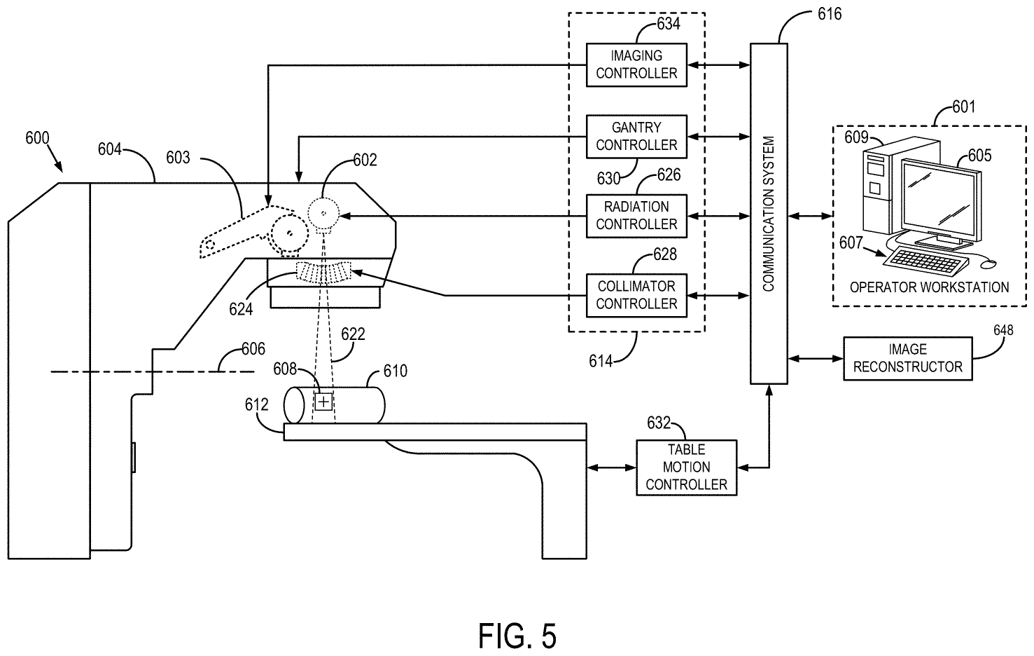

Referring to FIG. 5, a non-limiting example radiation therapy system 600 includes a therapeutic radiation source 602 and an on-board imaging source 603. The radiation source 602 and the on-board imaging source 603 may be housed in the same gantry system 604 or may be mounted orthogonally to the radiation source 602. The radiation therapy system 600 may include any suitable radiation treatment system, including image-guided radiation therapy (“IGRT”) systems, intensity-modulated radiation therapy (“IMRT”) systems such as intensity-modulated arc therapy (“IMAT”) and volumetric modulated arc therapy (“VMAT”) systems, an external beam radiotherapy delivery system, such as a linear accelerator (LINAC), proton radiotherapy systems, slice by slice photon radiotherapy systems (Tomotherapy), non-isocentric photon radiotherapy systems (Cyberknife), and isotope based radiotherapy systems (ViewRay and GammaKnife), and the like. In a non-limiting example, the radiation therapy system is a Truebeam STX linear accelerator with 6MV photons and HD-Multileaf Collimators (MLC). The treatment beam for the radiation therapy system can be composed of photons, neutrons, electrons, protons, heavy charged particles, or the like. Specific treatment plans can also be designed and delivered in order to evaluate key parameters of each radiotherapy system. Clinically relevant treatment plans can be prepared and utilized for end-to-end testing.

The on-board imaging source 603 may include an x-ray source, a Cone-Beam Computed Tomography (CBCT) system, a Computed Tomography (CT) system, a 4DCT system, a magnetic resonance imaging (MRI) system, and the like. Alternatively, the imaging may be performed by a separate diagnostic imaging system. Both the therapeutic radiation source 602 and imaging source 603 are attached adjacent each other and housed at the same end of a rotatable gantry 604, which rotates about a pivot axis 606. The rotatable gantry 604 allows either of the sources, 602 and 603, to be aligned in a desired manner with respect to a target volume 608 in an object 610 positioned on a table 612.

The rotation of the rotatable gantry 604, the position of table 612, and the operation of the sources, 602 and 603, are governed by a control mechanism 614 of the radiation therapy system 600. The control mechanism 614 includes a radiation controller 626 that provides power and timing signals to the radiation source 602, an imaging controller 634 that provides image acquisition instructions to imaging source 603, and receives image data therefrom, and a gantry motor controller 630 that controls the rotational speed and position of the gantry 604. The control mechanism 614 communicates with an operator workstation 601 and other parts of a network through communication system 616. An image reconstructor 648, receives sampled and digitized image data from the communication system 616 and performs high speed image reconstruction. The reconstructed image is applied as an input to a computer 609.

The computer 609 also receives commands and scanning parameters from an operator via a console that has a keyboard 607. An associated display 605 allows the operator to observe the reconstructed image and other data from the computer 609. The operator supplied commands and parameters are used by the computer 609 to provide control signals and information to the imaging controller 634, the radiation controller 626 and the gantry motor controller 630. In addition, the computer 609 operates a table motor controller 632 which controls the motorized table 612 to position the object 610 within the gantry 604.

Still referring now to FIG. 5, radiation source 602 produces a radiation beam, or “field,” 622, which in some forms may be conical or any other shape, emanating from a focal spot and directed toward an object 610. The radiation beam 622 may be initially conical and is collimated by a collimator 624 constructed of a set of rectangular shutter system blades to form a generally planar “fan” radiation beam 622 centered about a radiation fan beam plane. Each leaf of the collimator is constructed of a dense radio-opaque material such as lead, tungsten, cerium, tantalum, or related alloy.

A collimator control system 628 directed by a timer generating desired position signals provides electrical excitation to each electromagnet to control, separately, actuators to move each of the leaves of the MLC in and out of its corresponding sleeve and ray. The collimator control system 628 moves the leaves of the collimator 624 rapidly between their open and closed states to either fully attenuate or provide no attenuation to each ray. Gradations in the fluence of each ray, as needed for the fluence profile, are obtained by adjusting the relative duration during which each leaf is in the closed position compared to the relative duration during which each leaf is in the open position for each gantry angle. Alternatively, a physical cone or other structure may be used in place of the multi-leaf collimator.

The ratio between the closed and open states or the “duty cycle” for each leaf affects the total energy passed by a given leaf at each gantry angle, θ, and thus controls the average fluence of each ray. The ability to control the average fluence at each gantry angle, θ, permits accurate control of the dose provided by the radiation beam 622 through the irradiated volume of the object 610 by therapy planning methods to be described below. The collimator control system 628 also connects with a computer to allow program control of the collimator 624 to be described.

An image reconstructor 648, typically including a high speed array processor or the like, receives the data from the imaging controller 634 in order to assist in “reconstructing” an image from such acquired image data according to methods well known in the art. The image reconstructor 648 may also use post-radiation detector signals from a radiation detector to produce a tomographic absorption image to be used for verification and future therapy planning purposes as described in more detail below.

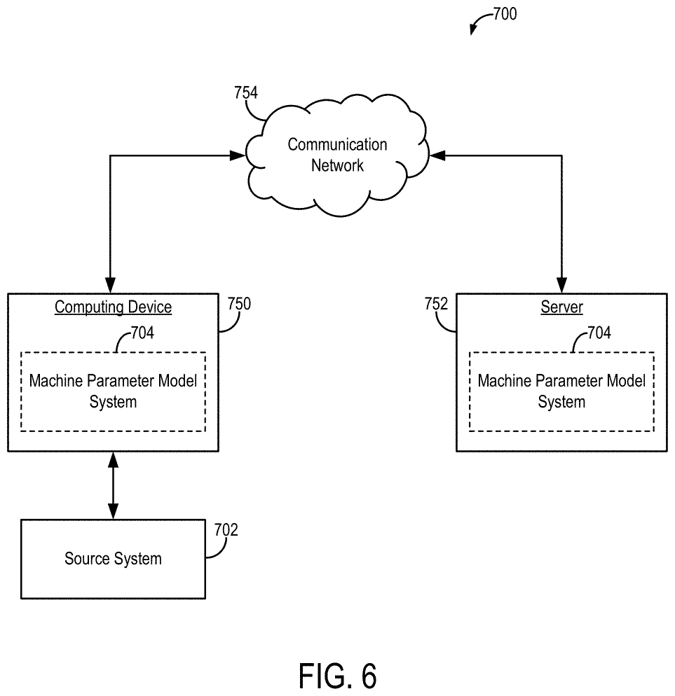

FIG. 6 shows an example 700 of a system for determining or predicting the MLC trajectory for a radiotherapy treatment plan and generating a machine parameter model using machine parameters from a source system 702 with machine learning or artificial intelligence and the like in accordance with some embodiments of the disclosed subject matter. As shown in FIG. 6, a computing device 710 can receive multiple types of data from a source system 702. In some configurations, computing device 710 can execute at least a portion of a machine parameter model system 704 to automatically generate a machine parameter model for determining MLC trajectory.

Additionally or alternatively, in some embodiments, computing device 710 can communicate information about machine parameter data received from source system 702 to a server 720 over a communication network 708, which can execute at least a portion of machine parameter model system 704 to automatically generate a machine parameter model. In such embodiments, server 720 can return information to computing device 710 (and/or any other suitable computing device) indicative of an output of machine parameter model system 704.

In some embodiments, computing device 710 and/or server 720 can be any suitable computing device or combination of devices, such as a desktop computer, a laptop computer, a smartphone, a tablet computer, a wearable computer, a server computer, a virtual machine being executed by a physical computing device, etc. In some configurations, machine parameter model system 704 can extract machine parameter data from the source system 702 and generate a model using a convolutional neural network (CNN) trained as a general classifier, and can perform a correlation analysis to calculate correlations between the features corresponding to the data and a database. In some embodiments, the labeled data can be used to train a classification model, such as a support vector machine (SVM), to generate a machine parameter model. In some embodiments, machine parameter model system 704 can provide features for machine parameter data to the trained classification model and can present a QA solution based on the output of the classification model.

In some embodiments, source system 702 can be any suitable source of data, such as a linear accelerator, radiotherapy system, IGRT, and the like. In some embodiments, source 702 can be local to computing device 710. For example, source 702 can be incorporated with computing device 710 (e.g., computing device 710 can be configured as part of a device for delivering radiotherapy, capturing and/or storing images). As another example, source 702 can be connected to computing device 710 by a cable, a direct wireless link, etc. Additionally or alternatively, in some embodiments, source 702 can be located locally and/or remotely from computing device 710, and can communicate data to computing device 710 (and/or server 720) via a communication network (e.g., communication network 708).

In some embodiments, communication network 708 can be any suitable communication network or combination of communication networks. For example, communication network 708 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 708 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, etc.

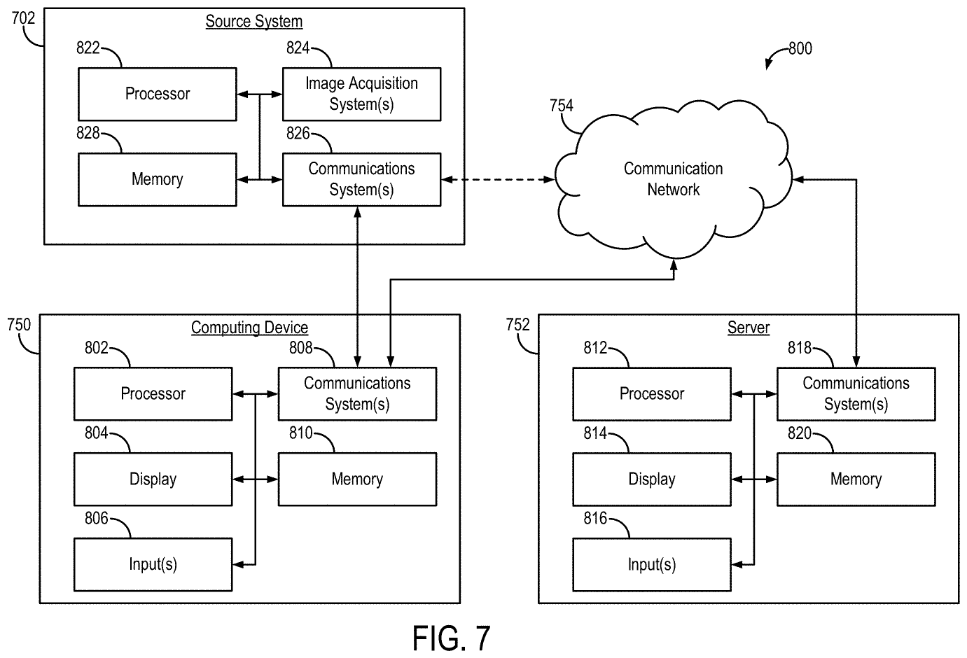

FIG. 7 shows an example 800 of hardware that can be used to implement source 702, computing device 710, and/or server 720 in accordance with some embodiments of the disclosed subject matter. As shown in FIG. 7, in some embodiments, computing device 710 can include a processor 802, a display 804, one or more inputs 806, one or more communication systems 808, and/or memory 810. In some embodiments, processor 802 can be any suitable hardware processor or combination of processors, such as a central processing unit (CPU), a graphics processing unit (GPU), etc. In some embodiments, display 804 can include any suitable display devices, such as a computer monitor, a touchscreen, a television, etc. In some embodiments, inputs 806 can include any suitable input devices and/or sensors that can be used to receive user input, such as a keyboard, a mouse, a touchscreen, a microphone, etc.

In some embodiments, communications systems 808 can include any suitable hardware, firmware, and/or software for communicating information over communication network 708 and/or any other suitable communication networks. For example, communications systems 808 can include one or more transceivers, one or more communication chips and/or chip sets, etc. In a more particular example, communications systems 808 can include hardware, firmware and/or software that can be used to establish a Wi-Fi connection, a Bluetooth connection, a cellular connection, an Ethernet connection, etc.

In some embodiments, memory 810 can include any suitable storage device or devices that can be used to store instructions, values, etc., that can be used, for example, by processor 802 to present content using display 804, to communicate with server 720 via communications system(s) 808, etc. Memory 810 can include any suitable volatile memory, non-volatile memory, storage, or any suitable combination thereof. For example, memory 810 can include RAM, ROM, EEPROM, one or more flash drives, one or more hard disks, one or more solid state drives, one or more optical drives, etc. In some embodiments, memory 810 can have encoded thereon a computer program for controlling operation of computing device 710. In such embodiments, processor 802 can execute at least a portion of the computer program to present content (e.g., QA results, dose delivery information, images, user interfaces, graphics, tables, etc.), receive content from server 720, transmit information to server 720, etc.

In some embodiments, server 720 can include a processor 812, a display 814, one or more inputs 816, one or more communications systems 818, and/or memory 820. In some embodiments, processor 812 can be any suitable hardware processor or combination of processors, such as a CPU, a GPU, etc. In some embodiments, display 814 can include any suitable display devices, such as a computer monitor, a touchscreen, a television, etc. In some embodiments, inputs 816 can include any suitable input devices and/or sensors that can be used to receive user input, such as a keyboard, a mouse, a touchscreen, a microphone, etc.

In some embodiments, communications systems 818 can include any suitable hardware, firmware, and/or software for communicating information over communication network 708 and/or any other suitable communication networks. For example, communications systems 818 can include one or more transceivers, one or more communication chips and/or chip sets, etc. In a more particular example, communications systems 818 can include hardware, firmware and/or software that can be used to establish a Wi-Fi connection, a Bluetooth connection, a cellular connection, an Ethernet connection, etc.

In some embodiments, memory 820 can include any suitable storage device or devices that can be used to store instructions, values, etc., that can be used, for example, by processor 812 to present content using display 814, to communicate with one or more computing devices 710, etc. Memory 820 can include any suitable volatile memory, non-volatile memory, storage, or any suitable combination thereof. For example, memory 820 can include RAM, ROM, EEPROM, one or more flash drives, one or more hard disks, one or more solid state drives, one or more optical drives, etc. In some embodiments, memory 820 can have encoded thereon a server program for controlling operation of server 720. In such embodiments, processor 812 can execute at least a portion of the server program to transmit information and/or content to one or more computing devices 710, receive information and/or content from one or more computing devices 710, receive instructions from one or more devices (e.g., a personal computer, a laptop computer, a tablet computer, a smartphone, etc.), etc.

In some embodiments, source 702 can include a processor 822, imaging components 824, one or more communications systems 826, and/or memory 828. In some embodiments, processor 822 can be any suitable hardware processor or combination of processors, such as a CPU, a GPU, etc. In some embodiments, imaging components 824 can be any suitable components to generate image data corresponding to one or more imaging modes (e.g., T1 imaging, T2 imaging, fMRI, etc.). An example of an imaging machine that can be used in conjunction with source system 702 can include a conventional MRI scanner (e.g., a 1.5 T scanner, a 3 T scanner), a high field MRI scanner (e.g., a 7 T scanner), an open bore MRI scanner, a CT system, an ultrasound scanner and the like.

Note that, although not shown, source 702 can include any suitable inputs and/or outputs. For example, source 702 can include input devices and/or sensors that can be used to receive user input, such as a keyboard, a mouse, a touchscreen, a microphone, a trackpad, a trackball, hardware buttons, software buttons, etc. As another example, source 702 can include any suitable display devices, such as a computer monitor, a touchscreen, a television, etc., one or more speakers, etc.

In some embodiments, communications systems 826 can include any suitable hardware, firmware, and/or software for communicating information to computing device 710 (and, in some embodiments, over communication network 708 and/or any other suitable communication networks). For example, communications systems 826 can include one or more transceivers, one or more communication chips and/or chip sets, etc. In a more particular example, communications systems 826 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, etc.

In some embodiments, memory 828 can include any suitable storage device or devices that can be used to store instructions, values, image data, etc., that can be used, for example, by processor 822 to: deliver radiotherapy, receive machine parameter data from a radiotherapy source system 702, control imaging components 824, and/or receive image data from imaging components 824; generate images; present content (e.g., MRI images, a user interface, etc.) using a display; communicate with one or more computing devices 710; etc. Memory 828 can include any suitable volatile memory, non-volatile memory, storage, or any suitable combination thereof. For example, memory 828 can include RAM, ROM, EEPROM, one or more flash drives, one or more hard disks, one or more solid state drives, one or more optical drives, etc. In some embodiments, memory 828 can have encoded thereon a program for controlling operation of source 702. In such embodiments, processor 822 can execute at least a portion of the program to generate a machine parameter model to one or more computing devices 710, receive information and/or content from one or more computing devices 710, receive instructions from one or more devices (e.g., a personal computer, a laptop computer, a tablet computer, a smartphone, etc.), etc.

In one configuration, a method, a system, or a computer-readable medium is provided as described herein, with the addition that differences between the actual machine parameters and values recorded in the trajectory file are compensated in the second radiotherapy plan by comparing machine parameters measured in routine machine QA tests and those recorded in trajectory files.

In one configuration, a method, a system, or a computer-readable medium is provided as described herein, with the addition that differences between calculated and actual dose due to limitations in the beam model are quantified using a plan robustness analysis. As an example, this plan robustness analysis may consist of a dosimetric analysis after re-calculating the dose using various beam models in which random perturbations have been introduced in the beam model parameters to account for the expected uncertainties in these values.

In one configuration, a method, a system, or a computer-readable medium is provided as described herein, with the addition that differences in actual and calculated dose due to potential variations in machine parameters at treatment delivery are quantified using a plan robustness analysis. In this case the plan robustness analysis may consist of a dosimetric analysis after introducing random perturbations to the treatment plan based on the expected uncertainty and reproducibility of these values.

The present disclosure has described one or more preferred embodiments, and it should be appreciated that many equivalents, alternatives, variations, and modifications, aside from those expressly stated, are possible and within the scope of the invention.

1. A method for performing a quality assurance (QA) test of a radiation therapy system, the method comprising:
a) generating a first radiotherapy treatment plan for irradiating a portion of a subject using the radiation therapy system;
b) subjecting the first radiotherapy treatment plan to a trained machine parameter determination model to generate predicted machine parameters for the radiation therapy system for delivery of a treatment plan;
c) generating a second radiotherapy treatment plan based on the predicted machine parameters; and
d) determining a dose effect to the subject based on the second radiotherapy treatment plan.

2. The method according to claim 1, wherein the machine parameter determination model has been trained using data from radiotherapy treatment machine parameter trajectory files.

3. The method according to claim 2, wherein the machine parameters include at least one of MLC velocity, MLC acceleration, MLC bank, control point number, monitor unit fraction, dose rate, gravity vector, gantry velocity, or gantry acceleration.

4. The method according to claim 2, wherein the machine parameter determination model is at least one of a machine learning model, artificial intelligence model, linear regression, decision tree, advanced decision-tree, ensemble algorithms, a neural network, or a convolutional neural network.

5. The method according to claim 2, wherein determining an independent dose delivery to the subject includes using an independent dose calculation carried out on the first radiotherapy treatment plan using the predicted machine parameters and the data from the radiotherapy treatment machine parameter trajectory files.

6. The method according to claim 1, further comprising determining a delivery discrepancy by:
determining trajectory file data for the second radiotherapy treatment plan;
determining MLC positioning accuracy for the second radiotherapy plan; and
determining a discrepancy between the trajectory file data for the second radiotherapy plan and the determined MLC positioning accuracy.

7. The method according to claim 6, further comprising incorporating determined delivery discrepancies into a dose-volume histogram (DVH) for the subject.

8. The method according to claim 6, further comprising testing MLC speed and positioning capability for the radiation therapy system.

9. The method according to claim 1, wherein the radiation therapy system is a linear accelerator.

10. A system for performing a quality assurance (QA) test of a radiation therapy system, the system comprising:
a radiotherapy system configured to provide radiation to a portion of a subject;
a computer system configured to:
a) generate a first radiotherapy treatment plan for irradiating the portion of the subject using the radiation therapy system;
b) subject the first radiotherapy treatment plan to a trained machine parameter determination model to generate predicted machine parameters for the radiation therapy system for delivery of a treatment plan;
c) generate a second radiotherapy treatment plan based on the predicted machine parameters; and
d) determine a dose effect to the subject based on the second radiotherapy treatment plan.

11. The system according to claim 10, wherein the machine parameter determination model has been trained using data from radiotherapy treatment machine parameter trajectory files.

12. The system according to claim 11, wherein the machine parameters include at least one of MLC velocity, MLC acceleration, MLC bank, control point number, monitor unit fraction, dose rate, gravity vector, gantry velocity, or gantry acceleration.

13. The system according to claim 11, wherein the machine parameter determination model is at least one of a machine learning model, artificial intelligence model, linear regression, decision tree, advanced decision-tree, ensemble algorithms, a neural network, or a convolutional neural network.

14. The system according to claim 11, wherein the computer system is further configured to determine an independent dose delivery to the subject using an independent dose calculation carried out on the first radiotherapy treatment plan using the predicted machine parameters and the data from the radiotherapy treatment machine parameter trajectory files.

15. The system according to claim 10, wherein the computer system is further configured to determine a delivery discrepancy by being configured to:
determine trajectory file data for the second radiotherapy treatment plan;
determine MLC positioning accuracy for the second radiotherapy plan; and
determine a discrepancy between the trajectory file data for the second radiotherapy plan and the determined MLC positioning accuracy.

16. The system according to claim 15, wherein the computer system is further configured to incorporate the determined delivery discrepancies into a dose-volume histogram (DVH) for the subject.

17. The system according to claim 15, wherein the computer system is further configured to test MLC speed and positioning capability for the radiation therapy system.

18. The system according to claim 10, wherein the radiation therapy system is a linear accelerator.

19. A non-transitory computer-readable medium having stored thereon instructions that, when executed by a processor, cause the processor to:
a) generating a first radiotherapy treatment plan for irradiating a portion of a subject using the radiation therapy system;
b) subjecting the first radiotherapy treatment plan to a trained machine parameter determination model to generate predicted machine parameters for the radiation therapy system for delivery of a treatment plan;
c) generating a second radiotherapy treatment plan based on the predicted machine parameters; and
d) determining a dose effect to the subject based on the second radiotherapy treatment plan.

20. The non-transitory computer-readable medium according to claim 19, wherein the machine parameter determination model has been trained using data from radiotherapy treatment machine parameter trajectory files.
